(19) United States
(12) Patent Application Publication
(10) Pub. No.: US 2025/0134816 A1
(43) Pub. Date: May 1, 2025

(54) DIRECTLY COMPRESSIBLE MANNITOL GRANULES

(71) Applicant: ROQUETTE FRERES, Lestrem (FR)

(72) Inventors: Baptiste BOIT, La Gorgue (FR); Antoine SALOME, Billy Berclau (FR); Fabrice BUQUET, Lestrem (FR); Philippe LEFEVRE, Haverskerque (FR); Nicolas TARLIER, Lestrem (FR)

(21) Appl. No.: 18/836,792

(22) PCT Filed: Feb. 8, 2024
(86) PCT No.: PCT/EP2023/025057

§ 371 Date: Aug. 8, 2024

(30) Foreign Application Priority Data
Feb. 8, 2022 (FR) 2201119
Oct. 28, 2022 (FR) 2211326

(51) Int. Cl. A61K 9/16; A61K 9/20

(52) U.S. Cl. CPC A61K 9/1623; A61K 9/1694; A61K 9/2018

(57) ABSTRACT

The invention relates to compressible mannitol granules and to a process for the preparation thereof. The invention also relates to their use for the preparation of tablets, in particular by direct compression.

### TECHNICAL FIELD

The invention relates to compressible mannitol granules and to a process for the preparation thereof. The invention also relates to their use for the preparation of tablets, in particular by direct compression.

### TECHNOLOGICAL BACKGROUND

Direct compression techniques allow the production of tablets containing precise amounts of active ingredients, at high speed, and at relatively low cost. This technology consists in strongly compressing a pulverulent composition in a die by means of two punches, in order to give it the shape of a tablet. The high pressure applied causes aggregation of the particles of the powder and produces a solid tablet.

These pulverulent compositions typically comprise excipients and active ingredients of interest, for example pharmaceutical, veterinary, cosmetic, food, nutraceutical, chemical or agrochemical interest.

The excipients most commonly found in direct compression are diluents, also referred to as “direct compression excipients” in this case, lubricants, (super-)disintegrants, flow agents, pH stabilizers, dyes, flavorings, surfactants.

In order to be capable of forming tablets, the pulverulent composition to be compressed always comprises at least one direct compression excipient and a lubricant. Direct compression excipients are the majority compounds of tablets and are responsible for the tableting capacity and flow properties of the powder. The most commonly used excipients are microcrystalline cellulose and lactose. The lubricant allows the newly formed tablets to be ejected from the dies. On some tablet presses, the lubricant is not mixed with the other powders, but sprayed directly onto the walls to be lubricated. It limits the stress created by the ejection and thus makes it possible to preserve the integrity of the tablets. The most commonly used lubricant is magnesium stearate, followed by calcium stearate and sodium stearyl fumarate.

The direct compression excipient should ensure that tablets are sufficiently hard to guarantee their integrity over time, particularly during handling, storage and transportation. This hardness can be increased by increasing the compression force applied to the powder to be compressed. The higher the compression force (Fc), the denser and harder the resulting tablet. When using mannitol powder as a direct compression excipient, however, there are limits to the use of high compressive forces to produce tablets of high hardness: there is a threshold Fc above which tablet lamination occurs. Lamination (including capping and flaking) takes the form of horizontal fracturing of the tablets, either in the middle or at the start of one of the two domed sections.

In the direct-compression mannitol market, PARTECK® M200 (MERCK) is currently the hardest available. However, as explained above, lamination phenomena appear as the Fc applied to PARTECK® M200 increases, so that certain hardnesses cannot be achieved. It would be advantageous to have a mannitol powder that could be used to obtain even harder tablets. This would make it possible, for example, to increase the quantity of non-compressible materials in the tablet formulation. It would also be advantageous to have a mannitol powder that does not easily laminate, in order to give greater freedom as regards the compression parameters used, and in particular to avoid the pre-compression step, which is impossible to carry out when the compression press is not equipped therewith. Non-sensitivity to lamination also makes it possible to do away with the cylindrical shape and develop domed tablets that are easier to swallow-particularly for patients on long-term treatments- or tablets with attractive shapes (such as sun, star) in the pediatric and nutraceutical fields. This also speeds up tablet press output and thus productivity.

### SUBJECT MATTER OF THE INVENTION

The aim of the present invention is therefore to provide a mannitol powder with improved compression behavior, particularly under compression conditions compatible with industrial tablet production.

A particular aim of the present invention is to provide a mannitol powder that can be used to manufacture tablets with a high degree of hardness and/or that does not laminate easily.

The present invention aims to solve the above-mentioned problems by proposing a mannitol excipient that furthermore has the other properties required for a direct compression excipient, e.g. In terms of particle size, flow or dissolution.

### PRESENTATION OF THE INVENTION

The inventors have succeeded in developing mannitol granules with unique compression behavior.

These microcrystalline mannitol granules are characterized in that:
the mannitol has a crystalline β content greater than or equal to 90%;
they have a volume mean diameter D(4;3) greater than or equal to 90 μm and less than or equal to 400 μm;
they have an aerated density greater than or equal to 600 g/L;
they have a specific surface area greater than or equal to 0.50 m²/g.

As can be seen from the examples below, at compression speeds compatible with industrial tablet production, these mannitol granules do not laminate, even when applying extreme compression forces (Fc) of 25 kN. Conversely, PARTECK® M200 and PEARLITOL® SD laminate after 10 kN.

It was thus possible to obtain tablets with a hardness of around 250 N with the mannitol granules of the present disclosure, whereas with PARTECK® M200 the maximum hardness was 168 N (see FIG. 6). Indeed, even though PARTECK® M200 offers superior compressibility up to around 10 kN Fc, it nevertheless laminates at higher Fc. In the end, the mannitol granules of the present disclosure provide the highest hardnesses, owing to their resistance at high Fc.

The mannitol granules according to the present disclosure have a volume mean diameter D(4;3) suitable for use in direct compression of 60 to 400 μm. The inventors have even succeeded in obtaining granules in the 100-200 μm range, making them particularly suitable for most pharmaceutical uses. In fact, to guarantee homogeneous mixing of mannitol with any active ingredients present, it is recommended that mannitol and active ingredients have the same particle size. However, most pharmaceutical active ingredients have a particle size of 100-200 μm.

The mannitol granules according to the present disclosure have a lower electrostatic charge than that obtained with PEARLITOL® 200SD, good flowability and low friability, preferably are not friable (Examples, section B.), making it an excellent candidate for use as a container filler, e.g. for capsules, sachets and logs (logs are single-dose sachets with a longitudinal, usually tubular shape, commonly referred to as “stickpacks”), or for use in continuous processes, which require continuous powder mixing and dosing steps. In addition, the mannitol granules according to the present disclosure have a pleasant, slightly sweet taste and a good dissolution profile. These qualities are particularly sought-after for use in sachets or logs, where the powder to be ingested is in direct contact with the oral cavity.

The mannitol granules according to the present disclosure can be used as a compression excipient in standard tablets as well as in other tablets, for example in orodispersible tablets, typically in combination with at least one (super-)disintegrant (Examples, section C.).

The mannitol granules according to the present disclosure also have very interesting properties for use as a filler in wet or dry granulation (Examples, section D.).

These mannitol granules can be obtained by a continuous process of spray granulation in a fluidized air bed of a mannitol solution, wherein:
the granulator bed temperature is greater than or equal to 30° C. and less than or equal to 70° C.; and
a fraction of the mannitol is recycled.

This process gives the particles excellent mechanical strength. Its productivity is good, and there is little variability in the final properties compared with other processes, particularly those carried out in batch mode.

In addition to obtaining mannitol granules with improved compressibility, the process of the invention can produce powders with a low fines content (see FIG. 4B). One hypothesis is that this provides the additional advantage that mannitol granules behave consistently from batch to batch. A percentage of fines expressed by weight will be perceived as low, but the reality of the quantity of fines by number is totally different. However, these fines can generate numerous problems in compression: lack of flow, non-homogeneity, lubrication difficulties, press clogging and static electricity. Poor control of the fines content, or variations in it, can therefore have a negative influence on the compressive behavior of mannitol powder.

An additional advantage of the process of the invention is therefore the possibility of reducing these fines. In fact, simple sieving does not remedy this problem: mannitol has self-adhesive properties, particularly through static electricity, and clogs sieves very quickly. What is more, since a large quantity of fines remain stuck to the larger particles during sieving, these fines are not effectively eliminated. Thus, instead of eliminating them, their formation can be avoided in the process of the invention, while guaranteeing a satisfactory average size for the finished product.

### SUMMARY OF THE INVENTION

The invention thus has as its first object microcrystalline mannitol granules, characterized in that:
said mannitol has a β-crystalline content greater than or equal to 90%; and
they have a volume mean diameter D(4;3) greater than or equal to 90 μm and less than or equal to 400 μm; and
they have an aerated density greater than or equal to 600 g/L; and
they have a specific surface area greater than or equal to 0.50 m²/g.

Preferably, said mannitol granules have a crystalline p content greater than or equal to 95%.

Preferably, said mannitol granules have an aerated density greater than or equal to 610 g/L.

Preferably, said mannitol granules have a packed density greater than or equal to 650 g/L.

Preferably, said mannitol granules have a specific surface area greater than or equal to 0.60 m²/g.

The invention also relates to a pulverulent composition comprising the mannitol granules according to the present disclosure, and at least one other ingredient.

The invention also relates to a process for preparing tablets, comprising the direct compression of a pulverulent composition according to the present disclosure.

The object of the invention is also a tablet made up of the pulverulent composition according to the present disclosure, or capable of being obtained by, or obtained by the process for preparing tablets according to the invention.

Another object of the invention is the use of mannitol granules according to the present disclosure, as a direct compression excipient, as a filler for filling capsules, sachets or logs, and/or as a filler in powder shaping, for example by wet or dry granulation.

The invention also relates to a process for granulating mannitol, characterized in that it is a continuous process for granulation by spraying a solution of mannitol into a fluidized air bed, wherein:
the granulator fluidized bed temperature is greater than or equal to 30° C. and less than or equal to 70° C.; and
a fraction of the mannitol is recycled.

Preferably in said process, the recycling rate is 30 to 70% by weight of the product that is extracted from the granulator. It is preferably still greater than or equal to 35% by weight of the product extracted from the granulator.

Preferably in said process, the volume mean diameter D(4;3) of the recycled mannitol particles is greater than or equal to 20 μm and less than or equal to 150 μm. It is preferably still greater than 25 μm.

Preferably in said process, the sprayed mannitol solution has a dry matter by weight greater than or equal to 20%, and less than or equal to 50%.

### BRIEF DESCRIPTION OF THE DRAWINGS

Other features, details and advantages of the invention will appear from reading the following detailed description, and by analyzing the appended drawings, in which:

FIG. 1 shows an example diagram of a process according to the invention.

FIG. 2 is a characterization table of mannitol powders according to the invention and of comparative mannitol powders.

FIGS. 3A, 3B, 3C, 3D, and 3E are scanning electron microscope photographs of mannitol powders according to the invention (3A, 3B, 3C) and comparative mannitol powders (3D, 3E).

FIGS. 4A and 4B are particle size distributions by number of mannitol powders according to the invention (4B) and comparative mannitol powders (4A).

FIG. 5 shows compression curves for mannitol powders according to the invention, obtained on the KORSCH XP1 single-punch press.

FIG. 6 shows compression curves for mannitol powders according to the invention and for comparative mannitol powders, the tablets having been prepared at a rate of 25 tablets/min on a single-punch development press which simulates compression on an industrial rotary press (STYLCAM® 200R, MEDEL'PHARM).

FIG. 7 shows compression curves for mannitol powders according to the invention and for comparative mannitol powders, the tablets having been prepared at a rate of 40 tablets/min on a single-punch development press which simulates compression on an industrial rotary press (STYLCAM® 200R, MEDEL'PHARM).

### DETAILED DESCRIPTION OF THE INVENTION

### Mannitol Granules

The invention firstly relates to microcrystalline mannitol granules, characterized in that:
said mannitol has a β-crystalline content greater than or equal to 90%; and
they have a volume mean diameter D(4;3) greater than or equal to 90 μm and less than or equal to 400 μm; and
they have an aerated density greater than or equal to 600 g/L; and
they have a specific surface area greater than or equal to 0.50 m²/g.

The expression “mannitol granules” conventionally refers to mannitol particles which, when observed under an electron microscope at a magnification of, for example, ×100, appear variable in shape, in particular non-spherical, with an irregular surface.

At a magnification of ×3000, fine particles of fine agglomerated crystals are generally visible at the surface of the mannitol granules according to the disclosure.

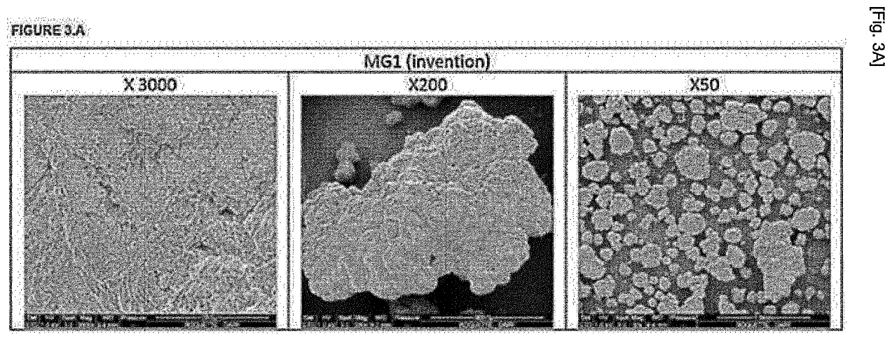

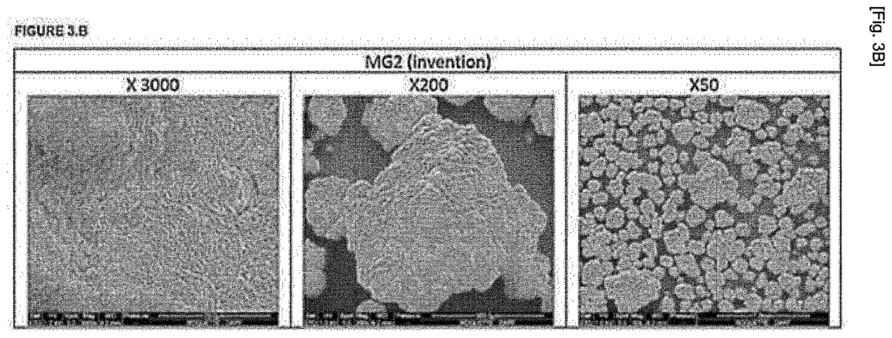

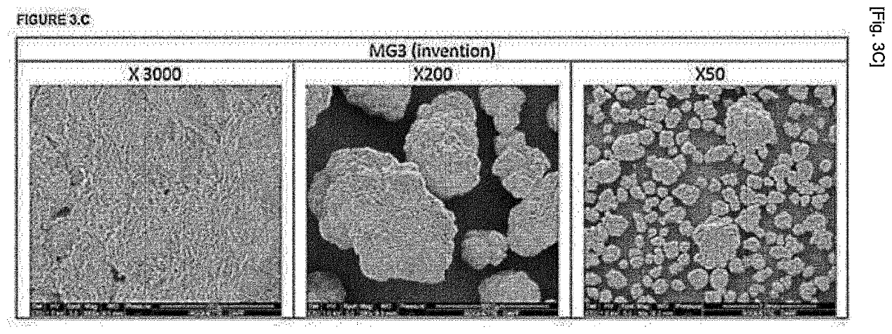

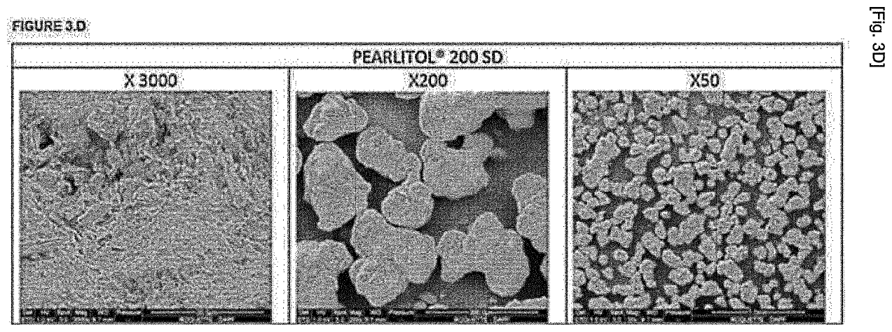

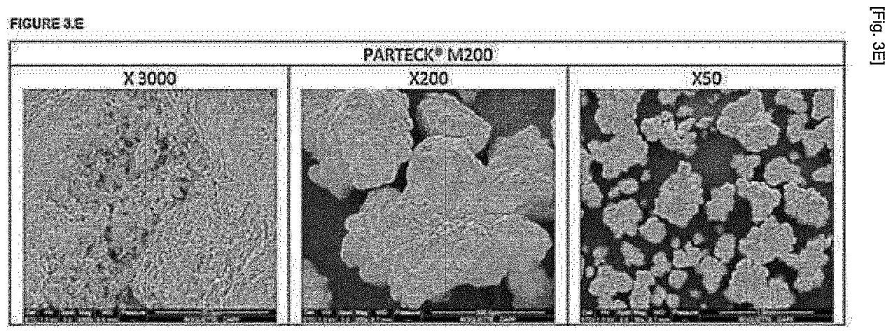

Preferably, the mannitol granules according to the present disclosure have a raspberry-like appearance (see FIGS. 3A, 3B and 3C for example, in particular at ×200 magnification). In contrast, the PEARLITOL® 200 SD particles shown in FIG. 3D, although also qualifying as mannitol granules, do not have this raspberry shape, and have a smoother surface than the mannitol granules according to the present disclosure.

Preferably, the mannitol granules according to the present disclosure exhibit little porosity at a magnification of, for example, ×3000.

The expression “microcrystalline” typically refers to a structure which, when observed under an electron microscope at a magnification for example of ×3000, substantially has microcrystals at its surface and very rarely any larger crystals. According to the present disclosure, a microcrystal may be defined as a crystal whose sum of length, width and thickness is less than 25 μm. The microcrystals can have very different shapes, from rounded shapes to elongate shapes. The granules according to the present disclosure have a microstructure that is preferably “non-filamentous”. In other words, the length-to-width ratio of the microcrystals present on the surface of the granules of the present disclosure is preferably lower than that observed for filaments. Indeed, even though needle-shaped crystals may be present, they are very much in the minority at the surface of the granules according to the disclosure. By comparison, patent U.S. Pat. No. 6,998,481 B2 presents a photo of a granule with a so-called filamentous texture because only needle-shaped microcrystals are visible. Finally, the microcrystals of the mannitol granules according to the present disclosure are generally non-oriented.

Under an electron microscope and at a magnification of ×3000, the mannitol granules according to the present disclosure are easily distinguished from the conventional crystalline powders of mannitol, composed of well-individualized macrocrystals, that are typically polyhedral, have a regular surface, have a substantially constant thickness but variable length and width, and are generally obtained by simple crystallization in water, from a solution supersaturated with mannitol. They are further distinguished from the mannitol powders obtained by agglomeration of a powder composed of mannitol macrocrystals. These granules are not microcrystalline in structure: the crystals, although no longer in individualized form, are still clearly visible and appear as sharp edges in these granules (examples of such granules are GRANUTOL™ F and S, photographs of which can be seen in the article Atsushi Kosufi et al. “Characterization of Powder- and Tablet Properties of Different Direct Compaction Grades of Mannitol Using a Kohonen Self-organizing Map and a Lasso Regression Model”. Journal of Pharmaceutical Sciences xxx (2020) 1-9). Note that the process described, for example, in the Example section below uses a mannitol powder (PEARLITOL® 160 C) composed of macrocrystals. However, this powder is only used as an initial primer, in very small quantities, and at the very start of the process. Thus, this macrocrystalline structure is not visible in the mannitol granules according to the present disclosure.

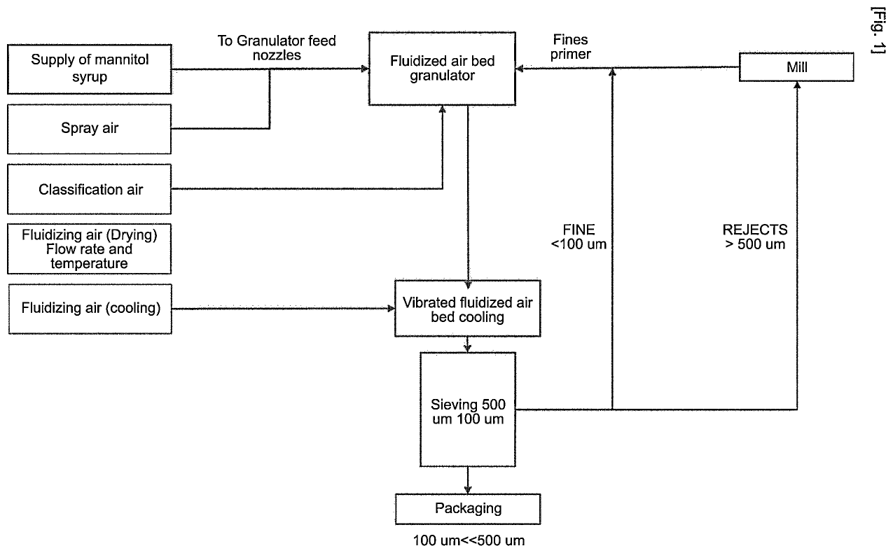

The mannitol granules according to the present disclosure also differ from mannitol powders obtained by single-effect spray drying (not employing a fluidized bed) of a mannitol solution, whose particles, although composed of microcrystalline mannitol, have a very smooth surface, are spherical or in the form of “deformed spheres” and of small diameter, generally between 10 and 50 μm (see for example Eva M. Littringer et al. “The morphology and various densities of spray dried mannitol”. Powder Technology 246 (2013) 193-200, and in particular, FIG. 1 p. 196). They are further distinguished from the mannitol powders obtained by melting/extrusion, which are made up of particles that are more compact and regular, which are in the form of more or less angular blocks, and which consist of generally oriented microcrystals.

The mannitol granules according to the disclosure can typically be obtained by a spray granulation process, wherein process mannitol granules are formed from a mannitol solution. Thus, alternatively, or additionally, microcrystalline mannitol granules according to the present disclosure can be defined by the fact that they are granulated, or obtained, or obtainable, by spray granulation, in particular by a fluidized bed spray-granulation process. Also, alternatively or additionally, mannitol granules according to the present disclosure may be defined by the fact that they are not obtained by single-effect atomization, and/or by fusion/extrusion, and/or by agglomeration of a powder, in particular composed of macrocrystals, and/or by dry granulation.

The mannitol granules according to the present disclosure are also characterized by the fact that the mannitol comprises at least 90% β-crystalline form. The crystalline polymorphism of the mannitol (crystalline forms and proportions) can be determined by the skilled person using infrared spectrometry or X-ray powder diffraction, preferentially X-ray powder diffraction. It is possible for example to perform this by carrying out the method as disclosed in the Examples section below. Preferably, the mannitol granules according to the present disclosure comprise at least 95% β-crystalline form, more preferably still at least 97%, more preferably still at least 98%, more preferably still at least 99%, more preferably still 100%. Note that these percentages of β-crystalline form are typically expressed based on the total represented by α, β, and δ crystalline forms.

The mannitol granules according to the present disclosure are also characterized by the fact that they have a volume mean diameter D(4;3) greater than or equal to 90 and less than or equal to 400 μm. This volume mean diameter D(4;3) is preferably greater than or equal to 100 μm, preferably greater than or equal to 120 μm, preferably greater than or equal to 140 μm, preferably greater than or equal to 150 μm, preferably greater than or equal to 160 μm, preferably greater than or equal to 170 μm, preferably greater than or equal to 180 μm, or even greater than or equal to 190 μm. It is preferably less than or equal to 350 μm, preferably less than or equal to 300 μm, preferably less than or equal to 250 μm, preferably less than or equal to 240 μm, preferably less than 230 μm, preferably less than or equal to 220 μm, preferably less than or equal to 210 μm, or even less than or equal to 200 μm. For example, it is about 190 μm, or about 200 μm. This volume mean diameter D(4;3) can in particular be determined by a person skilled in the art by means of a dry laser-diffraction particle size analyzer, for example according to the method as disclosed in the Examples section below.

Preferably, the mannitol granules according to the disclosure have a number D10 greater than or equal to 20 μm, preferably greater than or equal to 30 μm, preferably greater than or equal to 40 μm, preferably greater than or equal to 50 μm, preferably greater than or equal to 60 μm. It is generally less than or equal to 150 μm, or even less than or equal to 120 μm, or even less than or equal to 100 μm, or even less than or equal to 80 μm, or even less than or equal to 70 μm.

Preferably, the mannitol granules according to the disclosure have a number D50 greater than or equal to 30 μm, preferably greater than or equal to 50 μm, preferably greater than or equal to 70 μm, preferably greater than or equal to 80 μm, preferably greater than or equal to 90 μm. It is generally less than or equal to 200 μm, or even less than or equal to 150 μm, or even less than or equal to 140 μm, or even less than or equal to 130 μm, or even less than or equal to 120 μm, or even less than or equal to 110 μm, or even less than or equal to 100 μm.

Preferably, the mannitol granules according to the disclosure have a number D90 greater than or equal to 80 μm, preferably greater than or equal to 100 μm, preferably greater than or equal to 110 μm, preferably greater than or equal to 120 μm, preferably greater than or equal to 130 μm, preferably greater than or equal to 140 μm, preferably greater than or equal to 150 μm. It is generally less than or equal to 300 μm, or even less than or equal to 250 μm, or even less than or equal to 200 μm, or even less than or equal to 190 μm, or even less than or equal to 180 μm, or even less than or equal to 170 μm, or even less than or equal to 160 μm.

Preferably, the mannitol granules according to the disclosure have a volume D10 greater than or equal to 50 μm, preferably greater than or equal to 60 μm, preferably greater than or equal to 70 μm, preferably greater than or equal to 80 μm. It is generally less than or equal to 200 μm, or even less than or equal to 150 μm, or even less than or equal to 120 μm, or even less than or equal to 110 μm, or even less than or equal to 100 μm.

Preferably, the mannitol granules according to the disclosure have a volume D50 greater than or equal to 100 μm, preferably greater than or equal to 120 μm, preferably greater than or equal to 140 μm, preferably greater than or equal to 150 μm. It is generally less than or equal to 250 μm, or even less than or equal to 200 μm, or even less than or equal to 180 μm, or even less than or equal to 170 μm.

Preferably, the mannitol granules according to the disclosure have a volume D90 greater than or equal to 200 μm, preferably greater than or equal to 250 μm, preferably greater than or equal to 260 μm, preferably greater than or equal to 280 μm, preferably greater than or equal to 300 μm, preferably greater than or equal to 310 μm, preferably greater than or equal to 320 μm, preferably greater than or equal to 330 μm. It is generally less than or equal to 450 μm, or even less than or equal to 400 μm, or even less than or equal to 380 μm, or even less than or equal to 370 μm, or even less than or equal to 360 μm.

Recall that the values of D10, D50 and D90 by number are the sizes for which 10%, 50% and 90% by number, respectively, of the particles have a smaller particle size. The values of D10, D50 and D90 by volume are the sizes for which 10%, 50% and 90% by volume, respectively, of the particles have a smaller particle size.

The number or volume values of D10, D50 and D90 can in particular be determined by a person skilled in the art by means of a dry laser-diffraction particle size analyzer, for example according to the method as disclosed in the Examples section below.

The mannitol granules according to the disclosure are also characterized by the fact that they have an aerated density greater than or equal to 600 g/L. Preferably, this aerated density is greater than or equal to 610 g/L, preferably greater than or equal to 620 g/L. It is generally less than or equal to 750 g/L, or even less than or equal to 700 g/L, or even less than or equal to 650 g/L, or even less than or equal to 640 g/L. For example, it is about 630 g/L, or about 620 g/L.

Preferentially, the mannitol granules according to the present disclosure also have a packed density greater than or equal to 650 g/L, preferably greater than or equal to 700 g/L, preferably greater than or equal to 720 g/L, preferably greater than or equal to 730 g/L, preferably greater than or equal to 740 g/L. It is generally less than or equal to 850 g/L, or even less than or equal to 800 g/L, or even less than or equal to 790 g/L, or even less than or equal to 780 g/L, or even less than or equal to 770 g/L, or even less than or equal to 760 g/L. For example, it is about 750 g/L, or about 760 g/L.

This aerated density and packed density can be determined by a skilled person using the method recommended by the European Pharmacopoeia, in particular in accordance with Method 1: “measurement in a graduated cylinder” described in the European Pharmacopoeia 10.0, 2.9.34.

The mannitol granules according to the disclosure have a specific surface area greater than or equal to 0.50 m²/g, preferably greater than or equal to 0.60 m²/g, preferably greater than or equal to 0.70 m²/g, preferably greater than or equal to 0.75 m²/g, preferably greater than or equal to 0.80 m²/g, preferably greater than or equal to 0.90 m²/g, preferably greater than or equal to 1.00 m²/g, preferably greater than or equal to 1.10 m²/g, preferably greater than or equal to 1.20 m²/g, preferably greater than or equal to 1.30 m²/g. This specific surface area is generally less than or equal to 3.00 m²/g, or even less than or equal to 2.50 m²/g, or even less than or equal to 2.00 m²/g. For example, it is equal to about 1.30 m²/g, or equal to about 1.40 m²/g, or equal to about 1.50 m²/g. This specific surface area can be determined by a person skilled in the art, using the BET method, for example according to the method as disclosed in the Examples section below.

The mannitol granules according to the invention can also be characterized in that they are mannitol for direct compression or “directly compressible” mannitol. The term “direct compression excipient” is also conventionally used. The mannitol granules according to the disclosure can thus be compressed directly, that is, without any prior texturing or physical conversion treatment, for example such as a prior step of dry or wet granulation. It is understood that this means that the mannitol granules are capable of forming tablets of sufficient hardness, by direct compression, solely in the presence of an effective amount of lubricant. This “effective amount” is such that it effectively allows the formation of tablets, that is, typically, there is no adhesion, no binding, and the ejection force of the tablet from the press is less than 1000 Newtons, on a production for example of 10 tablets. This effective amount of lubricant generally does not exceed 3% by weight, relative to the total weight of the powder to be compressed. It is recalled that binding occurs when part of the material adheres to the die; this adhesion remains after the tablet has been ejected. Binding is visible on the tablet: vertical lines are present and correspond to the places where the product remained adhered to the die.

This ability to form satisfactory tablets can be determined for example by direct compression of a pulverulent composition consisting of the excipient to be tested and lubricant, for example magnesium stearate, so as to form convex tablets with a diameter of 10 mm a radius of curvature of 9 mm, and a weight of 400 mg. The tablets can be formed by means of a rotary press, or by means of a single-punch development press that simulates the compression on an industrial rotary press, for example such as the one used in the Examples section below. Press speed can be set to 25 tablets per minute or 40 tablets per minute. On MedelPharm's STYLCAM compression simulator, these speeds correspond to production rates of about 150,000 and 250,000 tablets per hour, respectively, on industrial rotary presses.

The hardness of the resulting tablets is measured using a hardness tester, for example such as the one used in the Examples section below. The hardness of the tablets prepared from the excipient to be tested solely in the presence of the lubricant, expressed in Newtons (N), denotes what is commonly referred to as the “tableting capacity” of the excipient.

According to a test referred to as “Test A” in the present disclosure, the tableting capacity of the excipient to be tested is determined as follows: convex 400 mg tablets with a diameter of 10 mm and a radius of curvature of 9 mm are prepared on a single-punch development press simulating compression on an industrial rotary press; the hardness of the tablets is then measured.

According to this test A, using a rate of 25 tablets per minute, the mannitol granules of the present disclosure preferably have a maximum tableting capacity (when Fc is varied, typically up to 25 kN) greater than or equal to 50 N, preferably greater than or equal to 100 N, preferably greater than or equal to 150 N, preferably greater than or equal to 200 N, preferably greater than or equal to 210 N, preferably greater than or equal to 220 N, preferably greater than or equal to 230 N, or even greater than or equal to 240 N. This maximum tableting capacity is generally less than or equal to 350 N, or even less than or equal to 300 N, or even less than or equal to 280 N, or even less than or equal to 270 N, or even less than or equal to 260 N, or even less than or equal to 250 N.

Still according to this test A, at a rate of 40 tablets per minute, this maximum tableting capacity is preferably greater than or equal to 50 N, preferably greater than or equal to 100 N, preferably greater than or equal to 150 N, preferably greater than or equal to 160 N, preferably greater than or equal to 170 N, preferably greater than or equal to 180 N, or even greater than or equal to 190 N. This maximum tableting capacity is generally less than or equal to 350 N, or even less than or equal to 300 N, or even less than or equal to 250 N, or even less than or equal to 230 N, or even less than or equal to 210 N, or even less than or equal to 200 N.

The mannitol granules according to the disclosure can also be characterized by the fact that they do not laminate at an Fc greater than 11 kN, preferably greater than or equal to 15 kN, preferably greater than or equal to 16 kN, preferably greater than or equal to 20 kN, preferably greater than or equal to 24 kN, preferably greater than or equal to 25 kN, the tablets for this lamination evaluation being produced according to the above-mentioned method for tableting capacity (convex tablets of 400 mg, 10 mm in diameter and with a radius of curvature of 9 mm, obtained on a single-punch development press which simulates compression on an industrial rotary press, using a rate of 25 or 40 tablets per minute). Preferably, the mannitol granules of the present disclosure do not laminate at all according to this test. It should be noted that, according to this test, forces in excess of 25 kN cannot be applied, as this would damage the punch. In fact, concave-shaped punches are more fragile and can withstand lower maximum Fc, since their edges are thinner.

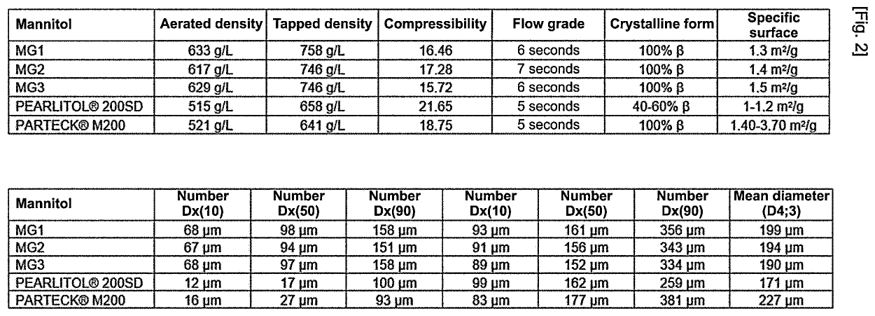

Preferably, the mannitol granules according to the present disclosure have a flow grade of 3 to 15 seconds, preferably less than or equal to 10 seconds, preferably less than or equal to 8 seconds. It is usually 5 seconds or more, or even 6 seconds or more. This flow grade can be determined by a skilled person according to the method recommended by the European Pharmacopoeia, for example the reference method described in “European Pharmacopoeia 7.0, 2.9.16, “Flow”, with equipment according to FIG. 2.9.16.-2”.

Preferably, the mannitol granules according to the present disclosure are not friable. This friability can be determined by a skilled person according to the method recommended by the European Pharmacopoeia, for example according to the reference method described in the European Pharmacopoeia 10.0, April 2012:20941 “2.9.41, Friability of Granules and Spheroids”. It is possible for example to perform this by carrying out the method as disclosed in the Examples section below.

Preferably, the mannitol granules according to the present disclosure have an electrostatic charge of less than 10.0 nC/g, preferably less than or equal to 8.0 nC/g, preferably less than or equal to 7.0 nC/g, preferably less than or equal to 6.0 nC/g, preferably less than or equal to 5.0 nC/g. This electrostatic charge can be determined by a person skilled in the art, using the GRANUCHARGE™ instrument, for example according to the method as disclosed in the examples below.

The mannitol granules according to the present disclosure are mannitol, but these granules may comprise other ingredients, in small amounts, and as long as this does not contravene the properties sought in the present invention. Examples of other ingredients are: binders such as polyvinylpyrrolidone (PVP), carboxymethylcellulose (CMC), hydroxypropylmethylcellulose (HPMC), cellulose-based derivatives, acacia gum, gelatin, starch derivatives such as maltodextrins, tragacanth gum; minerals; carbohydrates such as sugars and sugar alcohols other than mannitol; food additives, dyes; nutraceutical, pharmaceutical, veterinary or cosmetic active ingredients; preservatives; stabilizers. Preferentially, the content of other ingredients in the granules, in particular the content of carbohydrates other than mannitol, is less than 15.0%, preferentially less than 10.0%, preferentially less than 5.0%, preferentially less than 2.0%, preferentially less than 1.0%, even preferentially less than 0.5%; these percentages being expressed by weight relative to the total weight of granules.

Most preferentially, the mannitol granules according to the present disclosure are free of other ingredients. In the latter case, this means that the granules consist only of mannitol and residual impurities. It should be noted in these regards that the mannitol according to the present disclosure preferentially has a richness in mannitol, in particular in D-mannitol, greater than 95.0% by dry weight, preferentially greater than 96.0%, preferentially greater than 97.0%, preferentially greater than 97.5%, preferentially greater than 98.0%, preferentially greater than 98.5%, most preferentially greater than 99.0%, the rest being residual impurities typically arising from the production of the mannitol. The impurities typically comprise the substances associated with the mannitol, in particular sorbitol, maltitol and isomalt, reducing sugars, nickel, heavy metals. Their contents can be determined by a person skilled in the art for example according to the methods recommended by the European Pharmacopoeia (for example the method described in the reference document “Mannitol, January 2014:0559”).

Preferably, the mannitol granules according to the present disclosure have a mass loss with desiccation of 0.00 to 0.50% by weight. This mass loss with desiccation is preferentially less than or equal to 0.40% by weight, preferentially less than or equal to 0.30%. It is generally greater than or equal to 0.05%, or even greater than or equal to 0.10%, or even greater than or equal to 0.15%, or even greater than or equal to 0.20%. This mass loss with desiccation can be determined by the skilled person, for example using the “Karl Fischer” method, which is well known to the skilled person.

### Fluidized Air Bed Spray Granulation Process

Another object of the present invention is a mannitol granulation process, particularly useful for the manufacture of the mannitol granules disclosed above, characterized in that it is a continuous process for granulation by spraying a solution of mannitol into a fluidized air bed, wherein:
the granulator fluidized bed temperature is greater than or equal to 30° C. and less than or equal to 70° C.; and
a fraction of the mannitol is recycled.

The granulation process is a spray granulation process. Unlike “agglomeration” processes, in spray granulation the material to be granulated is in liquid form (not powder), in this case in the form of a mannitol solution. The granulated particles come from drying this solution on a primer obtained by recycling a fraction of the granulated particles previously obtained.

It is also a continuous process. Conventionally, this means that the final product is collected continuously, as long as the system is supplied with mannitol solution.

Preferably, the process according to the disclosure does not involve “exogenous” mannitol powder, that is, other than that generated by the mannitol solution, with the exception of negligible quantities of mannitol powder that may be employed at process start-up for initial priming.

The temperature of the fluidized bed is greater than or equal to 30° C. and less than or equal to 70° C. It is preferably greater than or equal to 40° C., and even more preferably greater than or equal to 45° C. It is preferably less than or equal to 60° C., and even more preferably less than or equal to 55° C. For example, it is about 47° C. or about 51° C.

The fluidization air flow rate is typically chosen to have a linear velocity in the bed of between 1.0 and 2.0 m/s, preferably around 1.5 m/s.

The fluidizing air temperature is typically set to control the fluidized bed temperature. For example, it is greater than or equal to 100° C., and less than or equal to 150° C. For example, it is greater than or equal to 110° C., or even greater than or equal to 120° C. For example, it is less than or equal to 140° C. For example, it is around 130° C.

Preferably, a circular fluidized bed is used.

Preferably, the sprayed mannitol solution has a dry matter by weight greater than or equal to 20%, preferably greater than or equal to 30%, preferably greater than or equal to 35%. It is preferably less than or equal to 50%, preferably less than or equal to 45%, preferably less than or equal to 40° C. For example, it is chosen in a range from about 38 to about 40%.

Preferably, the sprayed mannitol solution is maintained at a temperature (“feed temperature”) that keeps the mannitol in solution. Preferably, this feed temperature is greater than or equal to 70° C., preferably greater than or equal to 75° C., preferably greater than or equal to 80° C., preferably greater than or equal to 85° C. It is preferably less than or equal to 100° C., preferably less than or equal to 95° C., preferably less than or equal to 90° C. It is for example equal to about 88° C.

Preferably, spraying is carried out by means of dual-fluid spray nozzle(s). Their number is conventionally adapted to the size of the fluidized bed. These nozzles can be placed either at the top (“top spray”) or bottom (“bottom spray”) of the fluidized bed.

Preferably, the solution feed rate is greater than or equal to 300 kg/h/m² of fluidized bed, and less than or equal to 400 kg/h/m² of fluidized bed. It is preferably greater than or equal to 310 kg/h/m² of fluidized bed, more preferably greater than or equal to 320 kg/h/m² of fluidized bed. It is preferably less than or equal to 380 kg/h/m² of fluidized bed, preferably less than or equal to 350 kg/h/m² of fluidized bed, more preferably less than or equal to 340 kg/h/m² of fluidized bed. For example, it is equal to about 330 kg/h/m² of fluidized bed.

Preferably, the spray pressure is greater than or equal to 1.0 bar and less than or equal to 4.0 bar. It is preferably greater than or equal to 1.5 bar, preferably greater than or equal to 2.0 bar, preferably greater than or equal to 2.5 bar. It is preferably less than or equal to 3.5 bar, more preferably less than or equal to 3.0 bar.

Preferably, the spraying air temperature is greater than or equal to 20° C. and less than or equal to 100° C. It is preferably less than or equal to 80° C., preferably less than or equal to 60° C., preferably less than or equal to 50° C., preferably less than or equal to 40° C., preferably less than or equal to 30° C. It is for example equal to about 25° C.

Since the process is a continuous one involving recycling, a fraction of mannitol granules is continuously extracted from the granulator.

Preferably, particles with a size greater than or equal to 50 μm, preferably greater than or equal to 80 μm, more preferably greater than or equal to around 100 μm are extracted. It is understood that smaller particles are still extracted, insofar as the means used for this extraction (e.g. a classifier) generally do not enable a clean cut to be obtained.

Preferably, this extraction is carried out by means of an air classifier discharge tube. The particle size threshold for classification is set by the classification air flow rate. Preferably, the classification air flow rate is chosen so as to have a linear velocity in the tube of between 2.0 and 5.0 m/s, preferably between 3.0 and 4.5 m/s and preferably between 3.3 and 3.8 m/s.

Preferably, the process according to the disclosure comprises a step for cooling the mannitol granules after extraction from the granulator. This step is preferably carried out using a vibrated fluidized air bed.

The process according to the disclosure comprises recycling granulated mannitol particles, in particular fines. It is understood that the recycled fraction is reintroduced in dry form, that is, the granulated mannitol particles are not resolubilized in solution but directly re-injected into the granulator bed. The recycled particles typically come from mannitol extracted from the granulator. They are recycled as is, or after grinding. The few fine particles transported with the outgoing air flow can also be reintroduced into the recycling system, and are generally in very small quantities compared with the particles originating from the mannitol extracted, for example, with the aid of the classifier.

Preferably, the mannitol extracted from the granulator undergoes a separation step for particles considered to be too fine, and/or a separation step for particles considered to be too coarse, preferably both. Thus typically, the particles considered to be too fine will feed the recycling system without the need for grinding. The particles considered to be too coarse are fed into the recycling system after grinding. Preferably, this separation is carried out after a cooling step, in particular as disclosed above.

Preferably, when the system is running continuously and in steady state, the recycling rate is 30 to 70% by weight of the product extracted from the granulator. Preferably, this recycling rate is greater than or equal to 35%, preferably greater than or equal to 40%. It is preferably less than or equal to 65%, preferably less than or equal to 60%, preferably less than or equal to 55%.

Preferably, the average diameter of the recycled mannitol particles is smaller than the average diameter of the particles ultimately desired. The volume mean diameter D(4;3) of the recycled mannitol particles is preferably greater than or equal to 20 μm and less than or equal to 150 μm. It is preferably greater than 25 μm, preferably greater than 50 μm, preferably greater than 75 μm, preferably greater than or equal to 80 μm. It is preferably less than or equal to 150 μm, preferably less than or equal to 140 μm, preferably less than or equal to 130 μm, preferably less than or equal to 120 μm.

Preferably, this separation is achieved by means of one or more sieves. Preferably, the cut-off threshold used for particles considered to be too fine is greater than or equal to 50 μm and less than or equal to 150 μm. It is preferably greater than or equal to 80 μm, more preferably greater than or equal to 90 μm. It is preferably less than or equal to 130 μm, more preferably less than or equal to 110 μm. This cut-off threshold is, for example, about 100 μm. The fraction that passes is typically and preferably introduced into the recycling system as is (without being ground). Preferably, the cut-off threshold used for particles considered to be too coarse is greater than or equal to 400 μm and less than or equal to 800 μm. It is preferably greater than or equal to 450 μm. It is preferably less than or equal to 700 μm, preferably less than or equal to 600 μm, preferably less than or equal to 550 μm. This cut-off threshold is, for example, about 500 μm. The retained fraction is typically and preferably introduced into the recycling system after grinding.

Preferably, these sieving operations are carried out in series. Preferably, the sieves are arranged from highest to lowest cut-off. Thus preferably, sieving to separate particles considered to be too coarse is carried out before sieving to separate particles considered to be too fine.

The mannitol remaining after separation of particles considered too coarse and too fine is collected, and can be conditioned.

Preferably, an air mill with integrated classifier is used for grinding, whose settings (air flow rate, plate speed, classifier/selector speed) enable a size of fines to be obtained that ensures system balance. The size of the ground particles is typically smaller than the desired final particle size. Preferably, the volume mean diameter D(4;3) of the ground particles is as defined above for the recycled fraction.

Preferably, the process comprises an initial priming step, to enable granulation to start. For this step, powdered mannitol is introduced into the fluidized bed. Typically, between half and all of the bed is filled with this mannitol powder. Preferably, this amount of mannitol is less than or equal to 300 kg/m² of fluidized bed. Note that this “exogenous” mannitol represents a very small amount of mannitol compared with the total amount of mannitol used. For example, 200 to 600 kg of powder are used to produce 7 tons of mannitol granules over 24 hours. Typically, the amount of mannitol used for initial priming is preferably 10% or less of the weight of mannitol granules produced, and will tend toward 0% after several days of continuous production. In the following examples, crystallized mannitol powder (composed of mannitol macrocrystals) is used for initial priming. However, it is also possible to use textured mannitol, such as atomized or granulated mannitol.

In the case where the mannitol granules according to the disclosure comprise other ingredients than mannitol, the granulation process according to the disclosure includes the use of these other ingredients, which can be introduced in dry form into the granulator chamber, for example via the recycling system or an additional inlet, and/or in the form of a suspension and/or solution, for example via the sprayed mannitol solution.

In a preferred embodiment, the granulation process comprises:
i1) preparing a mannitol solution;
i2) spraying the mannitol solution in a fluidized bed granulator, the temperature of the fluidized bed being greater than or equal to 30° C. and less than or equal to 70° C., until mannitol particles are obtained;
i3) optionally, cooling the mannitol particles from step i2), preferably in a fluidized air bed;
i4) sieving the mannitol particles from step i2) or step i3) to obtain:
   mannitol granules with a volume mean diameter D(4;3) greater than or equal to 90 μm and less than or equal to 400 μm, and, optionally:
   mannitol particles with a diameter of less than 130 μm, preferably less than 110 μm, preferably less than 100 μm (particles which may also be referred to as “fines”), and/or
   mannitol particles with a diameter greater than 400 μm, preferably greater than 450 μm, preferably greater than 500 μm (particles which may also be referred to as “rejects”);
i5) recycling the fines from step i4) to the fluidized bed granulator of step i2);
i6) grinding the rejects from step i4) and recycling them into the granulator from step i2).

Preferably, the mannitol granules of step i4) conform to the mannitol granules of the present disclosure.

Preferably, the recycling rate is as defined above. In particular, 30 to 70% by weight of the mannitol particles from step i2) are recycled according to step i5) and step i6).

Preferably, at least 35% by weight of the mannitol particles obtained from step i2) are recycled according to step i5) and step i6).

Preferably, the process also comprises an initial priming step i0), wherein the bed of the fluidized air granulator is fed with mannitol powder at process start-up. Typically, between half and all of the bed is filled with this mannitol powder.

Preferably, the volume mean diameter D(4;3) of the recycled mannitol particles (comprising fines and ground rejects) is as defined above, in particular greater than 25 μm.

### Pulverulent Composition

The object of the present invention is also a pulverulent composition, in particular a pulverulent composition for direct compression, comprising the mannitol granules according to the disclosure and at least one other ingredient. The object of the present invention is also a pulverulent composition, in particular a pulverulent composition for direct compression, comprising the mannitol granules obtained or obtainable according to the granulation process according to the disclosure, and at least one other ingredient.

This pulverulent composition preferentially consists of:
30.0 to 99.9% of mannitol granules according to the present disclosure;
0.1 to 70.0% of ingredients other than the mannitol granules according to the disclosure;
   the percentages being expressed by weight, and their sum being equal to 100%.

Examples of other ingredients are typically:
direct compression excipients or diluents other than the mannitol of the invention, for example (i) directly compressible sugar alcohols such as the directly compressible forms of sorbitol, maltitol, xylitol, isomalt, lactitol, erythritol, or mannitol other than that according to the invention, (ii) directly compressible sugars such as the directly compressible forms of sucrose, dextrose, dextrates, lactose, allulose (iii) of the microcrystalline cellulose, (iv) directly compressible minerals;
lubricants;
dispersants or disintegrants, such as sodium starch glycolate, cross-linked carboxymethylcellulose, cross-linked polyvinylpyrrolidone (PVP), starches;
granulating agents such as polyvinylpyrrolidone, cellulose derivatives, acacia gum, dextrose, gelatin, maltodextrins, starches, starch derivatives, tragacanth gum;
food additives, such as flavorings, acidifying agents;
dyes such as mineral dyes, pigments or soluble dyes;
flow agents (e.g. silica dioxide), or anti-sticking agents (e.g. talc);
active ingredients, in particular pharmaceutical, veterinary, nutraceutical or cosmetic active ingredients.

Disintegrants are excipients whose role is to accelerate disintegration of the tablet, thus dispersing the active ingredient in water, digestive juices or, in the case of orodispersible tablets, in the oral cavity. They ensure rapid availability of active ingredients, while offering satisfactory rheological properties. So-called “super-disintegrants” are disintegrants that can be used at even lower concentrations than native starch. Examples of super-disintegrants include sodium starch glycolate, cross-linked carboxymethylcellulose and cross-linked PVP.

Preferentially, the pulverulent composition in accordance with the disclosure has a content of mannitol granules according to the disclosure of greater than or equal to 20%, preferably of greater than or equal to 30%, preferably of greater than or equal to 40%, these percentages being expressed in weight relative to the total weight of the pulverulent composition. This content of mannitol granules is generally less than or equal to 99%, or even less than or equal to 90%, or even less than or equal to 80%, or even less than or equal to 70%.

Generally, the pulverulent composition as disclosed has a lubricant content of 0.1 to 3.0%, preferably 0.2 to 3.0%, preferably 0.5 to 3.0%, preferably 1.0 to 3.0%, preferably 1.0 to 2.0%, these percentages being expressed by weight relative to the total weight of the pulverulent composition.

In an advantageous embodiment, particularly in the case of a composition for the manufacture of orodispersible tablets, the pulverulent composition as disclosed comprises a disintegrant, preferably a super-disintegrant.

The pulverulent compositions disclosed herein can be used to manufacture tablets. They can also be filler compositions, for example capsule and/or sachet and/or log filler compositions.

They can also be compositions designed for shaping by wet or dry granulation, for example dry granulation by slugging or roller compaction.

### Tablet Preparation

The present invention also relates to a process for preparing tablets, comprising the direct compression of a pulverulent composition according to the present disclosure, preferentially using a rotary press.

### Tablets

The object of the present invention is also a tablet made up of the pulverulent composition according to the disclosure, or capable of being obtained by, or obtained by the process for preparing tablets by direct compression according to the disclosure.

Conventionally, “tablet” is intended in the present disclosure to mean a solid preparation obtained by direct compression of a pulverulent composition. The tablet can be for example for food, pharmaceutical, cosmetic, nutraceutical use. They can be tablets for sucking, chewing, swallowing, orodispersible tablets or effervescent tablets. These tablets may be intended for humans, adults or children, or for animals. They may also be tablets for chemical or agrochemical use. These tablets may be single-layer or multi-layer tablets. In the present disclosure, the tablets preferentially have curved, or in other words convex, shape.

Preferably, the tablet according to the disclosure has a hardness greater than or equal to 50 N, preferably greater than or equal to 75 N, preferably greater than or equal to 100 N, preferably greater than or equal to 150 N, preferably greater than or equal to 200 N, preferably greater than or equal to 210 N, preferably greater than or equal to 220 N, preferably greater than or equal to 230 N, or even greater than or equal to 240 N, or even greater than or equal to 250 N, or even greater than or equal to 300 N, or even greater than or equal to 350 N. This hardness is generally less than or equal to 450 N, or even less than or equal to 400 N.

In an advantageous embodiment, the tablets are for use in individuals with swallowing difficulties, and/or for use in children and/or the elderly, and/or for use in individuals suffering from dysphagia.

### Use of the Mannitol Granules

Another object of the present invention is the use of mannitol granules according to the present disclosure, as a direct compression excipient, as a filler for filling capsules, sachets or logs, and/or as a filler in powder shaping, for example by wet or dry granulation.

Another object of the present invention is the use of mannitol granules according to the disclosure for the preparation of tablets, and/or for filling capsules, sachets or logs, and/or in continuous processes, e.g. comprising continuous powder mixing and/or dosing, e.g. in a continuous tablet, capsule, log or sachet preparation process, and/or in powder shaping processes, e.g. by wet or dry granulation, e.g. by slugging or roller compacting. Preferably, the granules according to the disclosure are used as a filler.

Another object of the present invention is the use of mannitol granules obtained or obtainable according to the granulation process according to the disclosure, as a direct compression excipient, as a filler for filling capsules, sachets or logs, and/or as a filler in powder shaping, for example by wet or dry granulation.

In the present disclosure, the quantities of ingredients are generally expressed as percentages by weight. Unless otherwise indicated, these weights are quantities of ingredients as is, in their powdered or oily form. The powdery ingredients generally contain small quantities of water (also called % moisture or “mass loss with desiccation”) and some impurities.

In contrast, in the present disclosure, when reference is made to a dry weight, this refers to anhydrous weights.

The invention will be better understood with the aid of the following examples, which are intended to be illustrative and non-limiting.

### EXAMPLES

### A. Preparation of Mannitol Powders

Several mannitol granule powders were prepared by spray granulation in a fluidized air bed.

The process was carried out as shown in FIG. 1. The fluidized-bed granulator (AGT type, diameter 1700 mm) was fed with a 38% dry matter mannitol solution. At the start of the process, the fluidized bed of the granulator was filled with 600 kg of mannitol powder (mannitol crystals with an average diameter of around 120 μm (PEARLITOL® 160 C). The sprayed solution was granulated in a circular fluidized bed. Sieving was carried out continuously on a three-deck ALLGAIER TSM 2000/3 tumbler screen equipped with a 100 μm screen with ultrasonic heads and a 500 μm screen. Particles smaller than 100 μm (considered to be too fine) were recycled directly, while particles larger than 500 μm (considered to be too coarse) were ground on a PAS 500 POITTEMILL-like mill and then reintroduced into the granulator chamber via the recycling system. The 100-500 μm cut was continuously collected to obtain the finished product.

The process conditions are presented in Table 1.

TABLE 1

| | Test 1 - MG1 | | | |
|---|---|---|---|---|
| | Test 2 - MG2 | | Test 3 - MG3 | |
| Number of Nozzles | 3 | | 3 | |
| Nozzle position | Top Spray | | Top Spray | |
| Feed rate | 750 kg/h (that is, | | 750 kg/h (that is, | |
| | 330 kg/h/m² of bed) | | 330 kg/h/m² of bed) | |
| Dry feed matter | 38% | | 38% | |
| Feed temperature | 88° | C. | 88° | C. |
| Spray pressure | 2.5 | bar | 2.8 | bar |
| Spray air temperature | 25° | C. | 25° | C. |
| Fluidizing air flow | 11500 | Nm³/h | 11500 | Nm³/h |
| Fluidization air temperature | 130° | C. | 130° | C. |
| Fluidized bed pressure drop | 150-250 | mmCE | 200-300 | mmCE |
| Fluidized bed temperature | 47° | C. | 51° | C. |
| Classification air flow rate | 400-450 | Nm³/h | 400-450 | Nm³/h |
| Grinder air flow rate | 3500 | Nm³/h | 3500 | Nm³/h |
| Plate speed | 1600 | rpm | 2200 | rpm |
| Classifier speed | 600 | rpm | 700 | rpm |
| Recycling rate by weight | 50-55% | | 40-45% | |
| of extracted product | | | | |
| Mean particle | 117 | μm | 86 | μm |
| diameter D(4; 3) of | | | | |

recycled fraction

The mannitol granules thus obtained after 48 h of continuous operation were characterized and compared with commercial and/or prior art mannitols.

### B. Characterization of Mannitol Powders

1. Determination of the densities. The aerated and packed densities of the test excipients were measured according to the European Pharmacopoeia method (European Pharmacopoeia 10.0, 2.9.34, Method 1: measurement in a graduated cylinder).

2. Determination of the specific surface area. The specific surface areas of the excipients to be tested were determined using a specific surface area analyzer (BECKMAN-COULTER, type SA3100), based on a nitrogen absorption test on the surface of the product subjected to the analysis, following the technique described in the article BET Surface Area by Nitrogen Absorption by S. BRUNAUER et al. (Journal of American Chemical Society, 60, 309, 1938). The BET analysis was carried out in 3 points.

3. Determination of crystalline forms. The ratio of crystalline forms was determined by X-ray powder diffraction: the crystalline forms of mannitol (alpha, beta and delta) were determined and quantified using an X-ray powder diffraction spectrometer with a copper anode tube (wavelength: 1.54 Å). Analysis was carried out continuously, from 5 to 60°, in reflection, using a rotating sample holder. The sample was compacted by hand and deposited in a flat layer on the sample holder. The crystalline forms of mannitol were determined by comparing the positions of the diffraction lines of the sample against the mannitol databases (Alpha ref. CSD1142501; Beta ref. CSD1142500; Delta ref. CSD 662815), corresponding to the references for the alpha, beta and delta forms of mannitol. Quantitative analysis was carried out using the Rietveld method with Topas V6 software (in the case of a Bruker spectrometer), using the fundamental parameters approach from structure files available on the COD (Crystallography Open Database) and CSD (Cambridge Structural Database) databases. The proportions of alpha, beta and delta polymorphs were determined by simulating a diffractogram based on the cif files of the reference products and after refining the various crystallographic parameters.

4. Granulometry: mean diameter D(4:3) and particle size distribution (by number or volume). The mean diameter D(4;3), D10, D50 and D90 (by number or volume) of mannitol powders were measured by dry laser diffraction, applying the Fraunhofer theory. The measurement was carried out using MASTERSIZER 3000 (MALVERN) dry process equipment (the dispersion accessory was the Aero S), following the manufacturer's technical manual and specifications. The dispersion attachment had a modular hopper with an opening of 0 to 4 mm. Work was done at zero pressure, 75% vibration and a hopper opening of 1.5 mm. The measurement range was 0.1 μm to 3500 μm. Obscuration was targeted between 8% and 10%. 2 measurements were taken for each sample. The result is the average of the two measurements. The volume data collected were: D(4;3)=mean diameter; D10, D50 and D90. The data recorded in number mode were the same diameters (except for D(4;3), which is always by volume).

5. Determination of the flow grade. The flow grade of the excipients to be tested was measured according to the method recommended by the European Pharmacopoeia (European Pharmacopoeia 7.0, 2.9.16, Flow; equipment according to FIG. 2.9.16.-2).

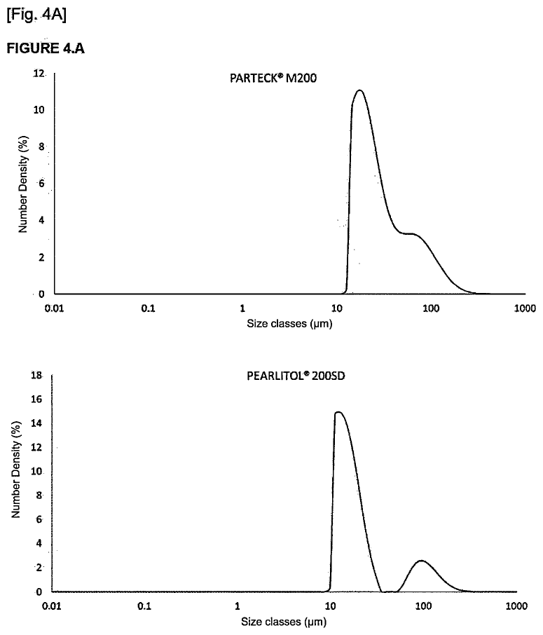

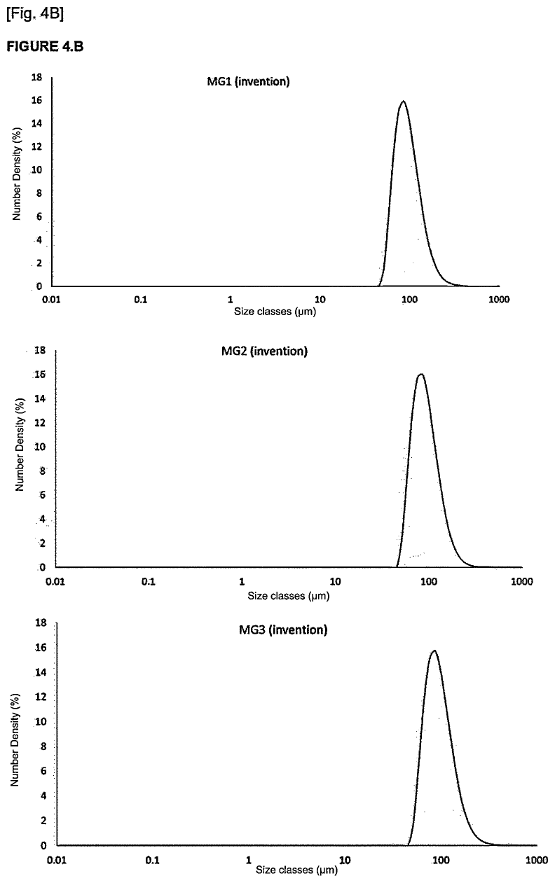

The results obtained are presented in the table in FIG. 2, as well as in FIG. 3 (microscopy) and FIG. 4 (number granulometry).

In a second step, the following parameters were measured on a new batch of mannitol granules according to the disclosure (MG4), obtained using the process according to the disclosure.

6. Electrostatic charge. Electrostatic charge was measured using the GRANUCHARGE™. The GRANUCHARGE™ instrument automatically and accurately measures the amount of electrostatic charge created inside a powder when flowing in contact with a selected material. The powder sample flows inside a vibrating V-shaped tube and falls into a Faraday cup connected to an electrometer. The electrometer measures the charge acquired by the powder as it flows through the V-shaped tube. For each experiment carried out with the GranuCharge, the following method was used: the free-flowing properties of the powder enabled the simple rotation of the beaker to feed the stainless steel tubing circuit. The quantity of powder used to perform a measurement can vary between 20-50 g. No recycling can be carried out after a measurement. Three different [temperature/humidity] pairs were selected to assess their influence on the electrostatic charge obtained. To avoid any dependence on temperature, we used absolute humidity as the reference value (water (in g) divided by the quantity of dry air (in kg)).

The absolute humidity values selected were:
5.3 g H₂O/kg dry air (Relative Humidity (RH)=35% and temperature (T°)=21° C.
6.0 g H₂O/kg dry air (RH=28% and T°=26° C.)
7.2 g H₂O/kg dry air (RH=46% and T°=21° C.)

All measurements were repeated 4 times to assess reproducibility.

The values obtained were 4.7 nC/g for MG4 mannitol granules, and 6.5 nC/g for PEARLITOL® 200SD.

7. Friability. Friability was determined using a friability tester (Friabimat SA-400, COPLEY), according to a method adapted from the European Pharmacopoeia 10.0, 2.9.41 “Friability of Granules and Spheroids”. Triplicate tests were carried out (10 g sample per test). The operating conditions were as follows: 400 oscillations per minute, for 240 seconds. The volume granulometry of the powder before and after passing through the friability tester was determined according to the method given in point 4. Two measurements were taken per sample. No air pressure was applied to avoid breakage. The particle size curves before and after passage through the friability tester were superimposable. In other words, granules conforming to the disclosure are not friable.

8. Dissolution time. The dissolution time of the test excipient was determined by measuring the time required to dissolve 5 g of powder immersed in 150 mL of demineralized water, at 20° C., using a magnetic stirrer rotating at 200 rpm. 45 mm bar. 250 mL tall beaker. The dissolution time obtained for MG4 mannitol granules was 30 seconds.

### C. Direct Compression Tests

MG1, MG2 and MG3 mannitol granules were first tested for their compression behavior on a KORSCH XP1 single-punch press. Extreme compression conditions were chosen to test the resistance of mannitol granules to lamination (convex tablets, compression speed of 60 tablets per minute).

The following pulverulent compositions were prepared: 98.8% by weight mannitol and 1.2% by weight magnesium stearate (Vegetal magnesium stearate, WIGA PHARMA GMBH) were mixed for 10 minutes in an epicycloidal mixer (TURBULA T2C, Willy A. Bachofen AG Maschinenfabrik, CH-4005 Basel), set at approximately 49 rotations per minute.

The powders were compressed using increasing compression forces, so as to form convex tablets having a diameter of 10 mm with a radius of curvature of 9 mm, and a weight of 400 mg.

The hardness of the tablets was measured using a hardness tester (PHARMATRON DT 50 503.0064).

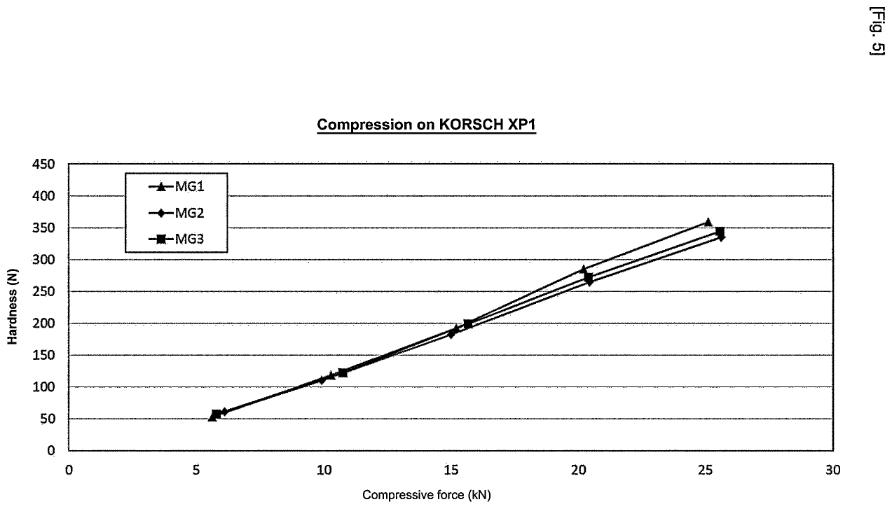

The results are presented in FIG. 5.

Despite extreme compression conditions, no lamination was observed. High hardnesses were obtained, in excess of 300 N, and even in excess of 350 N. It is also worth noting that the 3 batches have similar compression behavior: the process in accordance with the invention is robust and reproducible, and produces comparable powders from one batch to the next.

The mannitol granules were then tested for their compression behavior using a single-punch development press that simulates compression on an industrial rotary press, and compared with commercial and/or prior art mannitol powders.

Tablets were prepared in the same way as in the previous test, except for the press used.

The press used was a single-punch development press which simulates the compression on an industrial rotary press (STYLCAM® 200R, MEDEL'PHARM), controlled by ANALIS software, using the STYLCAM® standard profile. This press advantageously makes it possible to simulate the operation of industrial rotary presses. Two series of tests were carried out: in the first, the press was set to a speed of 25 tablets per minute. In a second test, the press was set to a speed of 40 tablets per minute. This latter speed corresponds to an industrial production rate of 250,000 tablets per hour.

The hardness of the tablets was measured using a hardness tester (PHARMATRON DT 50 503.0064).

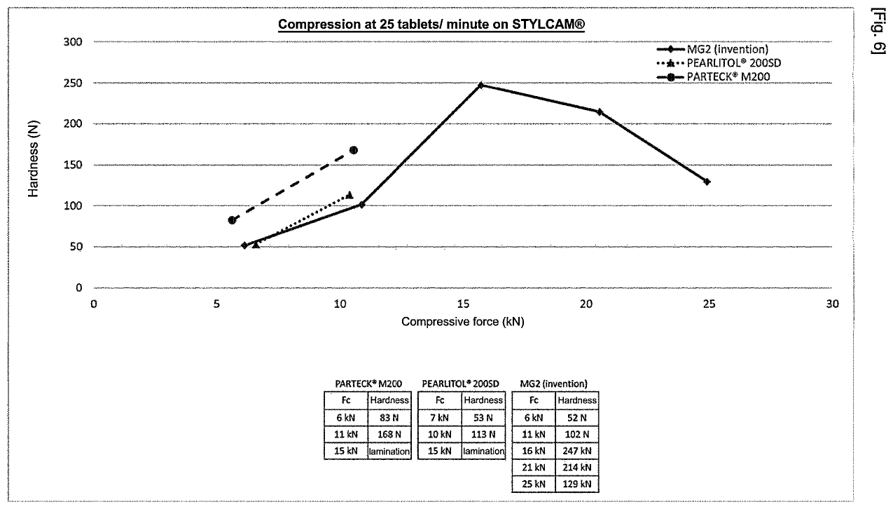

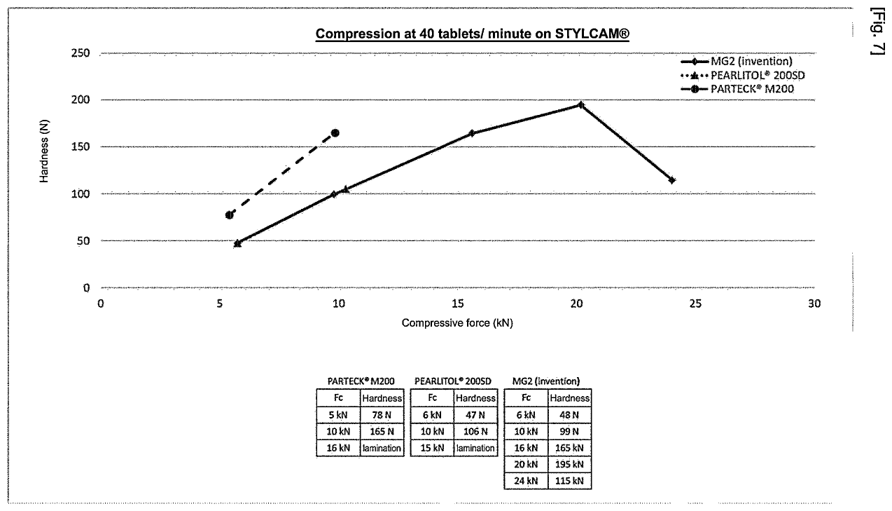

The results are shown in FIGS. 6 and 7.

At the two speeds tested, mannitol granules conforming to the disclosure did not laminate, whereas PARTECK® M200 and PEARLITOL® 200 SD laminated after 10-11 kN. Thus, the highest hardnesses, up to around 250 N (at 16 kN Fc), can be achieved with mannitol granules as disclosed.

Complementary compression tests were carried out on tablets containing an active pharmaceutical ingredient (results not shown here): the same trends were observed, that is, mannitol granules conforming to the disclosure did not laminate.

In this way, mannitol granules as disclosed enable the production of high-hardness tablets at high production speeds, compatible with industrial tablet production. Their resistance to lamination makes it possible to increase the quantities of non-compressible ingredients (typically active ingredient) in the tablets and/or to reduce tablet size. This is particularly advantageous for improving therapeutic compliance, especially in people with swallowing difficulties, such as children, the elderly and/or individuals suffering from dysphagia.

The disintegration time of tablets prepared at a rate of 25 tablets per minute was assessed using a method in accordance with the European Pharmacopoeia 7.1, April 2011:20901, “2.9.1. Disaggregation of tablets and capsules”. This disintegration time averaged 407 seconds (with a minimum of 398 seconds and a maximum of 418 seconds), well below the 15-minute limit generally acceptable for standard, “immediate-release” tablets.

The mannitol granules according to the disclosure have the qualities required for a compression excipient intended for an industrial production of tablets. They allow correct filling of the dies, that is, uniform and reproducible filling with a precise amount of powder, and flow correctly in the equipment used in direct compression. They are chemically and physically stable. They are sufficiently cohesive to allow them to be transported or to allow the preparation of mixtures. They do not hinder the bioavailability of the other ingredients of the powder and make it possible to obtain tablets that dissolve properly, in particular on contact with water. They allow homogeneous mixing of the ingredients of the composition and have a good absorption capacity. They allow the formulation of tablets with acceptable texture and taste, which is required when the tablet is intended to be ingested. They generate packaging and transport costs that adhere to commercial standards, that is, there is a good ratio between the weight of transported powder and the volume required for packaging this weight.

The mannitol granules according to the disclosure were then used in the preparation of orodispersible tablets.

The following pulverulent composition was prepared: 90.3% by weight mannitol granules, 0.5% by weight melatonin (active ingredient), 8% by weight crospovidone (cross-linked PVP) (KOLLIDON® CL) and 1.2% by weight magnesium stearate (Vegetal magnesium stearate, WIGA PHARMA GMBH). Mannitol granules, melatonin and crospovidone were mixed for 5 min in an epicycloidal mixer (TURBULA T2C, Willy A. Bachofen AG Maschinenfabrik, CH-4005 Basel), set at approximately 49 rotations per minute (rpm). Magnesium stearate was then added, and the mixture stirred for a further 5 minutes.

The powders were pressed to produce beveled tablets with a diameter of 10 mm (BEVEL EDGE D10 top and bottom punches ref. 2010060-5-1, die ref. 2010060-1), with a weight of about 380 mg, and a hardness of about 75 N. 10% pre-compression was applied.

The press used was a single-punch development press which simulates the compression on an industrial rotary press (STYLCAM® 200R, MEDEL'PHARM), controlled by ANALIS software, using the KORSCH XL 400 standard profile. The press was set to a speed of 20 tablets per minute, corresponding to an industrial output of 71,400 tablets per hour.

The disintegration rate of the resulting tablets was assessed in vitro and in vivo.

The in vitro disintegration time of the tablets was assessed using a method complying with the European Pharmacopoeia 7.1, April 2011:20901, “2.9.1. Disaggregation of tablets and capsules”.

The dissolution time obtained was 30 seconds in vivo, and 10 seconds in vitro, when no pre-compression force was applied. It was 35 seconds in vivo, and 10 seconds in vitro, when a pre-compression force of 1.45 kN was applied. These dissolution times are excellent for orodispersible tablets.

Thus, the mannitol granules according to the present disclosure can be used as a compression excipient in standard tablets as well as in other tablets, for example in orodispersible tablets, in particular when used in combination with at least one disintegrant, preferably a (super-)disintegrant.

### D. Use of Granules According to the Present Disclosure in Other Applications

The mannitol granules according to the present disclosure have a lower electrostatic charge than that obtained with PEARLITOL® 200SD, good flowability and are not friable (section B.), making it an excellent candidate for use as a container filler, e.g. for capsules, sachets and logs, or for use in continuous processes, which require continuous powder mixing and dosing steps. In addition, the mannitol granules according to the present disclosure have a pleasant, slightly sweet taste and a good dissolution profile. These qualities are particularly sought-after for use in sachets or logs, where the powder to be ingested is in direct contact with the oral cavity. The mannitol granules according to the disclosure are also suitable for use in wet or dry granulation.

In order to confirm the potential of granules conforming to the invention in these applications, the following tests were carried out.

### 1. Use for Capsule Filling

Mannitol granules conforming to the disclosure have been tested as a filler for capsule filling. The pulverulent composition used as filler consisted of 15% by weight active ingredient (propanolol), 64% by weight mannitol granules, 20% by weight partially gelatinized starch (LYCATAB® C, ROQUETTE) and 1% by weight magnesium stearate. The composition was prepared as follows: after 1 mm sieving of the active ingredient, mannitol granules and LYCATAB® C, mixing at 40 rpm for 10 minutes (T2F, TURBULA), addition of magnesium stearate after 355 μm sieving, mixing at 40 rpm for 1 minute. The parameters used for capsule filling were: FlexaLAB MG2 equipment (MG AMERICA); powder bed thickness 25 mm; dosing chamber filling height 13.5 mm; compression setting 1 mm; production speed 1000 capsules per hour; capsule size: size 1 (Capsugel®, Lonza); filling weight 270 mg; sampling interval 10 minutes. Capsule weight and disintegration time were measured at each sampling. Capsule disintegration time was determined on a disintegration tester (PTZ AUTO 3, Pharma Test) in demineralized water at 37° C.

Capsule weight was constant over time (350.38±1.30 mg), as was disintegration time (5.88±1.11 minutes). These results confirm that mannitol granules according to the disclosure are suitable for use as a filler in capsule filling.

### 2. Use in Wet Granulation

The mannitol granules were then tested for use in wet granulation (high-shear granulation). The pulverulent composition to be granulated was as follows: 15% by weight propanolol (active ingredient), 62% by weight mannitol granules, 3% by weight pregelatinized corn starch (LYCATAB® PGS, ROQUETTE), 20% by weight extra white corn starch. For granulation, 20% water was used, this percentage being expressed as the weight of water in relation to the weight of the pulverulent composition. Granulation was carried out as follows:
dry mixing in a high-shear granulator (Diosna): 300 g pulverulent composition sieved to 710 μm, mixing paddle speed 250 rpm, for 180 seconds.
water added to granulator: Schlick spray nozzles, 60 g water, flow rate about 20 g/min, for 3 minutes, mixing paddle speed 500 rpm, crumbler speed 1800 rpm.
granulation: mixing paddle speed 500 rpm, crumbler speed 1800 rpm, 3 minutes.
wet grinding in a conical mill (Quadro® Comil®): calibration grid 6350 μm, type of mesh: square, type of rotor: square, rotation speed 1000 rpm.
drying in a hot-air oven: overnight at 60° C.
dry grinding in a conical mill (Quadro® Comil®): calibration grid 1016 μm, type of mesh: rasp, type of rotor: square, rotation speed 1000 rpm.

The granulated product obtained was measured for particle size (method as per section B.4.), packed density, aerated density and moisture content.

The granulated product had a uniform particle size, with a volume D50 of around 200 μm. In contrast, the pulverulent composition before granulation had a bimodal distribution. The aerated density was 680 g/L and the packed density 780 g/L. The Carr and Hausner indices calculated from the density values were 12.59 and 1.14, respectively, indicating good flowability of the resulting granulated product. These results show that the granules according to the disclosure are suitable for use as a filler in wet granulation.

### 3. Use in Dry Granulation

The mannitol granules were then tested for use in dry granulation (“slugging”). The pulverulent composition to be granulated was as follows: 15% by weight propanolol (active ingredient), 54% by weight mannitol granules, 30% by weight microcrystalline cellulose (MICROCEL®102SD, ROQUETTE), 1% by weight magnesium stearate. The composition was prepared as follows: after passing through a 710 μm sieve, propanolol, mannitol granules and microcrystalline cellulose were mixed at 40 rpm for 10 minutes (T2F, TURBULA), magnesium stearate was added after 355 μm sieving, and mixed at 40 rpm for 1 minute. The pulverulent composition was then compressed using the following parameters: STYL'One Evolution press (MEDELPHARM), punches and die: round, chamfered EU-B, 16 mm (Natoli), press speed 25 rpm (Fette P2090 Euro B 54000 tablets/hour), compression force about 17 kN, tablet weight 1 g. The hardness of the tablets obtained was measured using a durometer (ST50, SOTAX), and was about 115 N. The tablets obtained were ground. The dry grinding parameters were as follows: calibration grid 1016 μm, mesh type: rasp, rotor type: square, rotation speed 3000 rpm.

The granulated product obtained was measured for particle size (method according to section B.4.), packed density and aerated density. The granulated product had a relatively uniform particle size, with a volume D50 of around 130 μm. The aerated density was 610 g/L and the packed density 780 g/L. The Carr and Hausner indices calculated from the density values were 21.97 and 1.28, respectively, indicating passable flowability of the resulting granulated product. These results show that the granules disclosed are suitable for use as fillers in dry granulation, although the process parameters can still be optimized in this case.

1. Microcrystalline mannitol granules, wherein:
said mannitol has a β-crystalline content greater than or equal to 90%; and
they have a volume mean diameter D(4;3) greater than or equal to 90 μm and less than or equal to 400 μm; and
they have an aerated density greater than or equal to 600 g/L; and
they have a specific surface area greater than or equal to 0.50 m²/g.

2. The microcrystalline mannitol granules according to claim 1, wherein they have a β-crystalline content greater than or equal to 95%.

3. The microcrystalline mannitol granules according to claim 1, wherein they have an aerated density greater than or equal to 610 g/L.

4. The microcrystalline mannitol granules according to claim 1, wherein they have a packed density greater than or equal to 650 g/L.

5. The microcrystalline mannitol granules according to claim 1, wherein they have a specific surface area greater than 0.60 m²/g.

6. A pulverulent composition comprising the microcrystalline mannitol granules according to claim 1, and at least one other ingredient.

7. A process for preparing tablets, comprising the direct compression of a pulverulent composition according to claim 6.

8. A tablet composed of the pulverulent composition according to claim 6.

9. A use of microcrystalline mannitol granules according to claim 1, as a direct compression excipient, as a filler for filling capsules, sachets or logs, and/or as a filler in powder shaping, for example by wet or dry granulation.

10. A process for granulating mannitol, wherein it is a continuous process for granulation by fluidized-air bed spraying of a mannitol solution, wherein:
the granulator fluidized bed temperature is greater than or equal to 30° C. and less than or equal to 70° C.; and
a fraction of the mannitol is recycled.

11. The process for granulating mannitol according to claim 10, wherein the recycling rate is 30 to 70% by weight of the product which is extracted from the granulator.

12. The process for granulating mannitol according to claim 11, wherein the recycling rate is greater than or equal to 35% by weight of the product which is extracted from the granulator.

13. The process for granulating mannitol according claim 10, wherein the volume mean diameter D(4;3) of the recycled mannitol particles is greater than or equal to 20 μm and less than or equal to 150 μm.

14. The process for granulating mannitol according to claim 13, wherein the volume mean diameter D(4;3) of the recycled mannitol particles is greater than 25 μm.

15. The process for granulating mannitol according to claim 10, wherein the sprayed mannitol solution has a dry matter by weight greater than or equal to 20%, and less than or equal to 50%.

16. A tablet obtainable by, or obtained by, the tablet preparation process according to claim 7.
